(19) United States
(12) Patent Application Publication
(10) Pub. No.: US 2009/0142554 A1
(43) Pub. Date: Jun. 4, 2009

(54) Yellow Azo Dyes for Ink Jet Printing

(76) Inventors: Gavin Wright, Manchester (GB); Rachel Anne James, Manchester (GB); Roy Bradbury, Merseyside (GB); Lynn Bradbury, Merseyside (GB)

Correspondence Address: MORGAN LEWIS & BOCKIUS LLP, 1111 PENNSYLVANIA AVENUE NW, WASHINGTON, DC, 20004, US

(21) Appl. No.: 12/083,832

(22) PCT Filed: Oct. 13, 2006
(86) PCT No.: PCT/GB2006/003807

§ 371 Date: Apr. 18, 2008

(30) Foreign Application Priority Data
Oct. 22, 2005 (GB) 0521549.6

(51) Int. Cl. C09D 11/02; C07D 213/84; C07D 213/73; C07D 213/81; C07D 403/12; C07D 215/22; B05D 5/00; B41M 5/00; G01D 11/00

(52) U.S. Cl. 428/195.1; 106/31.48; 546/289; 546/288; 546/308; 546/307; 544/212; 546/157; 427/256; 347/100

(57) ABSTRACT

A process for printing an image on a substrate by means of an ink-jet printer which comprises applying thereto a composition comprising a liquid medium and a mono-azo compound of Formula (1) and salts thereof:

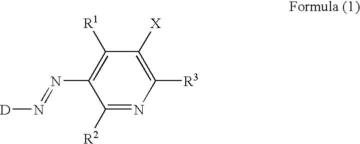

wherein:
D is an optionally substituted aryl group carrying at least one direct substituent selected from the group consisting of —SO₃H, —CO₂H, —PO₃H₂ or an optionally substituted nitrogen heteroaryl group carrying at least one direct substituent selected from the group consisting of —SO₃H, —CO₂H, —PO₃H₂;
R¹ is H: optionally substituted alkyl: amino: —NR⁴R⁵; wherein R⁴ is H, optionally substituted alkyl, optionally substituted alkoxy, optionally substituted acyl, optionally substituted aryl, optionally substituted heterocyclyl; and R⁵ is optionally substituted alkyl, optionally substituted alkoxy, optionally substituted acyl, optionally substituted aryl, optionally substituted heterocyclyl: —OR⁶; wherein R⁶ is H or optionally substituted alkyl: or —SR⁷; wherein R⁷ is optionally substituted alkyl;
R² and R³ are each independently preferably amino: —NR⁴R⁵; wherein R⁴ is H, optionally substituted alkyl, optionally substituted alkoxy, optionally substituted acyl, optionally substituted aryl, optionally substituted heterocyclyl; and R⁵ is optionally substituted alkyl, optionally substituted alkoxy, optionally substituted acyl, optionally substituted aryl, optionally substituted heterocyclyl: hydroxy —OR⁶; wherein R⁶ is H or optionally substituted alkyl: or —SR⁷; wherein R⁷ is optionally substituted alkyl;
X is H, or an electron withdrawing group, or X together with R³ forms an optionally substituted five or six membered carbocycle. Also compositions and inks, compounds and ink-jet printer cartridges.

This invention related to ink-jet printing processes, and compounds, compositions and inks, to printed substrates and to ink-jet printer cartridges.

Ink-jet printing is a non-impact printing technique in which droplets of ink are ejected through a fine nozzle onto a substrate without bringing the nozzle into contact with the substrate. The set of inks used in this technique typically comprise yellow, cyan, magenta and black inks.

With the advent of high-resolution digital cameras and ink-jet printers it is becoming increasingly common for consumers to print off photographs using an ink-jet printer. This avoids the expense and inconvenience of conventional silver halide photography and provides a print quickly and conveniently.

While ink-jet printers have many advantages over other forms of printing and image development there are still technical challenges to be addressed. For example, there are the contradictory requirements of providing ink colorants that are soluble in the ink medium and yet do not run or smudge excessively when printed on paper. The inks need to dry quickly to avoid sheets sticking together after they have been printed, but they should not form a crust over the tiny nozzle used in the printer. Storage stability is also important to avoid particle formation that could block the tiny nozzles used in the printer especially since consumers can keep an ink-jet ink cartridge for several months. Furthermore, and especially important with photographic quality reproductions, the resultant images should not fade rapidly on exposure to light or common oxidising gases such as ozone. One of the key factors in determining colour appearance of an ink-jet print is the chroma intensity of the component colours and small changes in this can have a profound impact on the quality of the image, this is especially true when the image is a photographic reproduction.

There are many thousands of known colorants and few have the characteristics which enable them to be used in ink-jet inks.

We have surprisingly found that certain azo pyridine compounds provide yellow colorants which are particularly suitable for use in ink-jet printing.

According to the present invention there is provided a process for printing an image on a substrate by means of an ink-jet printer which comprises applying thereto a composition comprising a liquid medium and a mono-azo compound of Formula (1) and salts thereof:

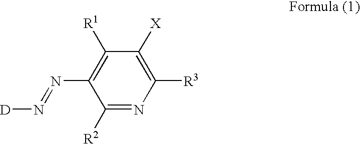

wherein:
D is an optionally substituted aryl group carrying at least one direct substituent selected from the group consisting of —SO₃H, —CO₂H, —PO₃H₂ or an optionally substituted nitrogen heteroaryl group carrying at least one direct substituent selected from the group consisting of —SO₃H, —CO₂H, —PO₃H₂;
R¹ is H: optionally substituted alkyl: amino: —NR⁴R⁵; wherein R⁴ is H, optionally substituted alkyl, optionally substituted alkoxy, optionally substituted acyl, optionally substituted aryl, optionally substituted heterocyclyl; and R⁵ is optionally substituted alkyl, optionally substituted alkoxy, optionally substituted acyl, optionally substituted aryl, optionally substituted heterocyclyl: —OR⁶; wherein R⁶ is H or optionally substituted alkyl: or —SR⁷; wherein R⁷ is optionally substituted alkyl;
R² and R³ are each independently preferably amino: —NR⁴R⁵; wherein R⁴ is H, optionally substituted alkyl, optionally substituted alkoxy, optionally substituted acyl, optionally substituted aryl, optionally substituted heterocyclyl; and R⁵ is optionally substituted alkyl, optionally substituted alkoxy, optionally substituted acyl, optionally substituted aryl, optionally substituted heterocyclyl: hydroxy —OR⁶; wherein R⁶ is H or optionally substituted alkyl: or —SR⁷; wherein R⁷ is optionally substituted alkyl;
X is H, or an electron withdrawing group, or X together with R³ forms an optionally substituted five or six membered carbocycle.

The ink-jet printer preferably applies the ink to the substrate in the form of droplets that are ejected through a small orifice onto the substrate. Preferred inkjet printers are piezoelectric ink-jet printers and thermal ink-jet printers. In thermal ink-jet printers, programmed pulses of heat are applied to the ink in a reservoir by means of a resistor adjacent to the orifice, thereby causing the ink to be ejected from the orifice in the form of small droplets directed towards the substrate during relative movement between the substrate and the orifice. In piezoelectric ink-jet printers the oscillation of a small crystal causes ejection of the ink from the orifice. Alternately the ink can be ejected by an electromechanical actuator connected to a moveable paddle or plunger, for example as described in International Patent Application WO00/48938 and International Patent Application WO00/55089.

The substrate is preferably paper, plastic, a textile, metal or glass, more preferably paper, an overhead projector slide or a textile material, especially paper.

Preferred papers are plain, treated or coated papers which may have an acid, alkaline or neutral character. Glossy papers are especially preferred.

Examples of commercially available treated papers include HP Premium Coated Paper, HP Photopaper™ (both available from Hewlett Packard Inc.); Stylus™ Pro 720 dpi Coated Paper, Epson Photo Quality™ Glossy Film, Epson Photo Quality™ Glossy Paper (all available from Seiko Epson Corp.); Canon HR 101 High Resolution™ Paper, Canon GP 201 Glossy™ Paper, Canon HG 101 and HG201 High GIOSS™ Film, Canon PR101 (all available from Canon); Kodak Premium™ Photopaper, Kodak Premium™ Inkjetpaper (available from Kodak); Konica lnkjet Paper QP™ Professional Photo Glossy, Konica Inkjet Paper QP™ Professional Photo 2-sided Glossy, Konica lnkjet Paper QP™ Premium Photo Glossy, Konica lnkjet Paper QP™ Premium Photo Silky (available from Konica).

Preferably when D is optionally substituted aryl it is optionally substituted phenyl or optionally substituted napthyl carrying at least one direct substituent selected from the group consisting of —SO₃H, —CO₂H, —PO₃H₂. More preferably D is optionally substituted phenyl or optionally substituted napthyl carrying at least one substituent selected from the group consisting of —SO₃H, —CO₂H especially —SO₃H.

In compounds of Formula (1) and salts thereof D is preferably optionally substituted phenyl or optionally substituted napthyl carrying 1 to 3-SO₃H substituents.

Examples of preferred phenyl and naphthyl groups represented by D are 2,5-disulfophenyl, 1,3,6-trisulfo-7-naphthyl, 2-sulfo-4-methoxyphenyl, 4-sulfophenyl.

When D is an optionally substituted nitrogen heteroaryl group then preferably nitrogen is the only heteroatom in the heteroaryl ring.

Preferably when D is an optionally substituted nitrogen heteroaryl group it is a 5 membered ring with preferably 2 or 3 nitrogen atoms in the ring, more preferably D is a 1,3,4-triazole carrying at least one direct substituent selected from the group consisting of —SO₃H, —CO₂H, —PO₃H₂.

R¹ is preferably H: optionally substituted C₁₋₄alkyl: amino: —NR⁴R⁵; wherein R⁴ is H, optionally substituted C₁₋₄alkyl, optionally substituted C₁₋₄alkoxy, optionally substituted C₁₋₄acyl, optionally substituted phenyl, optionally substituted heterocyclyl; and R⁵ is optionally substituted C₁₋₄alkyl, optionally substituted C₁₋₄alkoxy, optionally substituted C₁₋₄acyl, optionally substituted phenyl, optionally substituted heterocyclyl: —OR⁶; wherein R⁶ is H or optionally substituted C₁₋₄alkyl: or —SR⁷; wherein R⁷ is C₁₋₄alkyl.

It is especially preferred that R¹ is H, amino, NHR⁵ wherein R⁵ is optionally substituted C₁₋₄alkyl or optionally substituted C₁₋₄acyl.

R² and R³ are each independently preferably: amino; —NR⁴R⁵; wherein R⁴ is H, optionally substituted C₁₋₄alkyl, optionally substituted C₁₋₄alkoxy, optionally substituted C₁₋₄acyl, optionally substituted phenyl, optionally substituted heterocyclyl; and R⁵ is optionally substituted C₁₋₄alkyl, optionally substituted C₁₋₄alkoxy, optionally substituted C₁₋₄acyl, optionally substituted phenyl, optionally substituted heterocyclyl: —OR⁶; wherein R⁶ is H or optionally substituted C₁₋₄alkyl: or —SR⁷; wherein R⁷ is C₁₋₄alkyl.

It is especially preferred that R² and R³ are each independently: amino; NHR⁵ wherein R⁵ is optionally substituted C₁₋₄alkyl or optionally substituted C₁₋₄acyl; —OR⁶; wherein R⁶ is H or optionally substituted C₁₋₄alkyl.

When X is an electron withdrawing group, it is preferably selected from the group consisting of CN, CO₂H, CO₂R⁸, CON(R⁸)₂, COR⁸ and —SO₂N(R⁸)₂ wherein R⁸ is independently H, optionally substituted C₁₋₄alkyl, optionally substituted C₁₋₄alkoxy, optionally substituted C₁₋₄acyl, optionally substituted phenyl, optionally substituted heterocyclyl. When X is an electron withdrawing group it is particularly preferred that it is CN.

When X together with R³ forms an optionally substituted five or six membered carbocycle it is preferably an optionally substituted six membered carbocycle, more preferably a six membered carbocycle with a single hydroxyl substituent.

Optional substituents which may be present on R¹, R², R³, R⁴, R⁵, R⁶, R⁷, R⁸, X or D may be independently selected from: optionally substituted alkyl (preferably C₁₋₄-alkyl), optionally substituted alkoxy (preferably C₁₋₄-alkoxy), optionally substituted aryl (preferably phenyl), optionally substituted aryloxy (preferably phenoxy), optionally substituted heterocyclyl, polyalkylene oxide (preferably polyethylene oxide or polypropylene oxide), carboxy, phosphato, nitro, cyano, halo, ureido, —SO₂F, hydroxy, ester, —NR^{a}R^{b}, COR^{a}, —CONR^{a}R^{b}, —NHCOR^{a}, carboxyester, sulfone, and —SO₂NR^{a}R^{b}, wherein R^{a} and R^{b} are each independently H or optionally substituted alkyl (especially C₁₋₄-alkyl). Optional substituents for any of the substituents described above may be selected from the same list of substituents.

Compounds of Formula (1) and salts thereof are preferably free from fibre reactive groups. The term fibre reactive group is well known in the art and is described for example in EP 0356014 A1. Fibre reactive groups are capable, under suitable conditions, of reacting with the hydroxyl groups present in cellulosic fibres or with the amino groups present in natural fibres to form a covalent linkage between the fibre and the dye. As examples of fibre reactive groups preferably not present in the compounds of the first aspect of the present invention there may be mentioned aliphatic sulfonyl groups which contain a sulfate ester group in the beta-position to the sulfur atom, e.g. beta-sulfato-ethylsulfonyl groups, alpha, beta-unsaturated acyl radicals of aliphatic carboxylic acids, for example acrylic acid, alpha-chloro-acrylic acid, alpha-bromoacrylic acid, propiolic acid, maleic acid and mono- and dichloro maleic; also the acyl radicals of acids which contain a substituent which reacts with cellulose in the presence of an alkali, e.g. the radical of a halogenated aliphatic acid such as chloroacetic acid, beta-chloro and beta-bromopropionic acids and alpha, beta-dichloro- and dibromopropionic acids or radicals of vinylsulfonyl- or beta-chloroethylsulfonyl- or beta-sulfatoethyl-sulfonyl-endo-methylene cyclohexane carboxylic acids. Other examples of cellulose reactive groups are tetrafluorocyclobutyl carbonyl, trifluoro-cyclobutenyl carbonyl, tetrafluorocyclobutylethenyl carbonyl, trifluoro-cyclobutenylethenyl carbonyl; activated halogenated 1,3-dicyanobenzene radicals; and heterocyclic radicals which contain 1, 2 or 3 nitrogen atoms in the heterocyclic ring and at least one cellulose reactive substituent on a carbon atom of the ring.

When the compound of Formula (1) is in the form of a salt the preferred salts are alkali metal salts (especially lithium, sodium and potassium salts), ammonium and substituted ammonium salts and mixtures thereof. Especially preferred salts are sodium, potassium and lithium salts, salts with ammonia and volatile amines and mixtures thereof.

The compounds may be converted into a desired salt using known techniques. For example, an alkali metal salt of a compound may be converted into the ammonium or substituted ammonia salt by dissolving an alkali metal salt of the compound in water, acidifying with a mineral acid and adjusting the pH of the solution to pH 9 to 9.5 with ammonia or the amine and removing the alkali metal cations by dialysis or by use of an ion exchange resin.

The compounds of Formula (1) and salts thereof as described herein, may exist in tautomeric forms other than those shown in this specification. These tautomers are also included within the scope of the present inventions.

The compounds of Formula (1) may be prepared by diazotising a compound of formula D-NH₂ wherein D is as hereinbefore defined to give a diazonium salt and coupling the resultant diazonium salt with a compound of Formula (2) wherein X, R¹, R² and R³ are as hereinbefore defined:

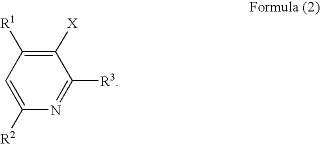

Diazotisations are preferably performed at a temperature of 0° C. to 10° C. Preferably diazotisations are performed in water, preferably at a pH below 7. Dilute mineral acid, e.g. HCl or H₂SO₄, may be used to achieve the desired pH conditions.

Reaction conditions are those generally used in the dyestuff art, for example as described in for example EP 0356080.

The liquid medium may comprise water, a mixture of water and organic solvent or organic solvent free from water.

Preferably the liquid medium comprises a mixture of water and organic solvent or organic solvent free from water.

When the liquid medium comprises a mixture of water and organic solvent, the weight ratio of water to organic solvent is preferably from 99:1 to 1:99, more preferably from 99:1 to 50:50 and especially from 95:5 to 80:20.

It is preferred that the organic solvent present in the mixture of water and organic solvent is a water-miscible organic solvent or a mixture of such solvents. Preferred water-miscible organic solvents include C₁₋₆alkanols, preferably methanol, ethanol, n-propanol, isopropanol, n-butanol, sec-butanol, tert-butanol, n-pentanol, cyclopentanol and cyclohexanol; linear amides, preferably dimethylformamide or dimethylacetamide; ketones and ketone-alcohols, preferably acetone, methyl ether ketone, cyclohexanone and diacetone alcohol; water-miscible ethers, preferably tetrahydrofuran and dioxane; diols, preferably diols having from 2 to 12 carbon atoms, for example pentane-1,5-diol, ethylene glycol, propylene glycol, butylene glycol, pentylene glycol, hexylene glycol and thiodiglycol and oligo- and poly-alkyleneglycols, preferably diethylene glycol, triethylene glycol, polyethylene glycol and polypropylene glycol; triols, preferably glycerol and 1,2,6-hexanetriol; mono-C₁₋₄-alkyl ethers of diols, preferably mono-C₁₋₄-alkyl ethers of diols having 2 to 12 carbon atoms, especially 2-methoxyethanol, 2-(2-methoxyethoxy)ethanol, 2-(2-ethoxyethoxy)-ethanol, 2-[2-(2-methoxyethoxy)ethoxy]ethanol, 2-[2-(2-ethoxyethoxy)-ethoxy]-ethanol and ethyleneglycol monoallylether; cyclic amides, preferably 2-pyrrolidone, N-methyl-2-pyrrolidone, N-ethyl-2-pyrrolidone, caprolactam and 1,3-dimethylimidazolidone; cyclic esters, preferably caprolactone; sulfoxides, preferably dimethyl sulfoxide and sulfolane. Preferably the liquid medium comprises water and 2 or more, especially from 2 to 8, water-miscible organic solvents.

Especially preferred water-miscible organic solvents are cyclic amides, especially 2-pyrrolidone, N-methyl-pyrrolidone and N-ethyl-pyrrolidone; diols, especially 1,5-pentane diol, ethyleneglycol, thiodiglycol, diethyleneglycol and triethyleneglycol; and mono-C₁₋₄-alkyl and C₁₋₄-alkyl ethers of diols, more preferably mono-C₁₋₄-alkyl ethers of diols having 2 to 12 carbon atoms, especially 2-methoxy-2-ethoxy-2-ethoxyethanol.

Examples of further suitable liquid media comprising a mixture of water and one or more organic solvents are described in U.S. Pat. No. 4,963,189, U.S. Pat. No. 4,703,113, U.S. Pat. No. 4,626,284 and EP-A-425,150.

When the liquid medium comprises organic solvent free from water, (i.e. less than 1% water by weight) the solvent preferably has a boiling point of from 30° to 200° C., more preferably of from 40° to 150° C., especially from 50 to 125° C. The organic solvent may be water-immiscible, water-miscible or a mixture of such solvents. Preferred water-miscible organic solvents are any of the hereinbefore-described water-miscible organic solvents and mixtures thereof. Preferred water-immiscible solvents include, for example, aliphatic hydrocarbons; esters, preferably ethyl acetate; chlorinated hydrocarbons, preferably CH₂Cl₂; and ethers, preferably diethyl ether; and mixtures thereof.

When the liquid medium comprises water-immiscible organic solvent, preferably a polar solvent is included because this enhances solubility of the dye in the liquid medium. Examples of polar solvents include C₁₋₄alcohols.

In view of the foregoing preferences it is especially preferred that where the liquid medium is organic solvent free from water it comprises a ketone (especially methyl ethyl ketone) and/or an alcohol (especially a C₁₋₄-alkanol, more especially ethanol or propanol).

The organic solvent free from water may be a single organic solvent or a mixture of two or more organic solvents. It is preferred that when the liquid medium is an organic solvent free from water it is a mixture of 2 to 5 different organic solvents. This allows a liquid medium to be selected that gives good control over the drying characteristics and storage stability of the ink.

Liquid media comprising organic solvent free from water are particularly useful where fast drying times are required and particularly when printing onto hydrophobic and non-absorbent substrates, for example plastics, metal and glass.

The liquid medium may of course contain additional components conventionally used in ink-jet printing inks, for example viscosity and surface tension modifiers, corrosion inhibitors, biocides, kogation reducing additives and surfactants which may be ionic or non-ionic.

Although not usually necessary, further colorants may be added to the ink to modify the shade and performance properties. Examples of such colorants include C.I.Direct Yellow 8; C.I.Direct Yellow 11; C.I.Direct Yellow 12; C.I.Direct Yellow 27; C.I.Direct Yellow 28; C.I.Direct Yellow 29; C.I.Direct Yellow 44; C.I.Direct Yellow 50; C.I.Direct Yellow 85; C.I.Direct Yellow 86; C.I.Direct Yellow 132; C.I.Direct Yellow 142; C.I.Direct Yellow 173; C.I.Acid Yellow 17; C.I.Acid Yellow 19; C.I.Acid Yellow 23; C.I.Acid Yellow 25; C.I.Acid Yellow 40; C.I.Acid Yellow 42; C.I.Acid Yellow 44; C.I.Acid Yellow 49; C.I.Acid Yellow 61; C.I.Acid Yellow 127; C.I.Acid Yellow 151; C.I.Acid Yellow 199 and C.I.Acid Yellow 219.

It is preferred that the composition according to the invention is ink suitable for use in an ink-jet printer. Ink suitable for use in an ink-jet printer is ink which is able to repeatedly fire through an ink-jet printing head without causing blockage of the fine nozzles.

Ink suitable for use in an ink-jet printer preferably has a viscosity of less than 20 cP, more preferably less than 10 cP, especially less than 5 cP, at 25° C.

The surface tension of ink suitable for use in an ink-jet printer is preferably in the range 20-65 dynes/cm, more preferably in the range 30-60 dynes/cm, at 25° C.

Ink suitable for use in an ink-jet printer preferably contains less than 500 ppm, more preferably less than 250 ppm, especially less than 100 ppm, more especially less than loppm in total of divalent and trivalent metal ions (other than any divalent and trivalent metal ions bound to a colorant of Formula (1) or any other component of the ink).

Preferably ink suitable for use in an ink-jet printer has been filtered through a filter having a mean pore size below 10 μm, more preferably below 3 μm, especially below 2 μm, more especially below 1 μm. This filtration removes particulate matter that could otherwise block the fine nozzles found in many ink-jet printers.

Preferably ink suitable for use in an ink-jet printer contains less than 500 ppm, more preferably less than 250 ppm, especially less than 100 ppm, more especially less than 10 ppm in total of halide, particularly chloride, ions.

Preferred compositions comprise:

(a) from 0.01 to 30 parts of dyes of Formula (1) and salts thereof; and

(b) from 70 to 99.99 parts of a liquid medium;

wherein all parts are by weight.

Preferably the number of parts of (a)+(b)=100.

The number of parts of component (a) is preferably from 0.1 to 20, more preferably from 0.5 to 15, and especially from 1 to 5 parts. The number of parts of component (b) is preferably from 80 to 99.9, more preferably from 85 to 99.5 and especially from 95 to 95 parts.

Preferably component (a) is completely dissolved in component (b). Preferably component (a) has a solubility in component (b) at 20° C. of at least 10%. This allows the preparation of liquid dye concentrates that may be used to prepare more dilute inks and reduces the chance of the dye precipitating if evaporation of the liquid medium occurs during storage.

The inks may be incorporated in an ink-jet printer as a high concentration yellow ink, a low concentration yellow ink or both a high concentration and a low concentration ink. In the latter case this can lead to improvements in the resolution and quality of printed images. Thus, the present invention also provides a composition (preferably an ink) where component (a) is present in an amount of 2.5 to 7 parts, more preferably 2.5 to 5 parts (a high concentration ink) or component (a) is present in an amount of 0.5 to 2.4 parts, more preferably 0.5 to 1.5 parts (a low concentration ink).

The pH of the composition is preferably from 4 to 11, more preferably from 7 to 10.

According to a second aspect of the invention there is provided a composition comprising a compound of Formula (1) and salts thereof, as defined in the first aspect of the invention, and a liquid medium that comprises a mixture of water and organic solvent or organic solvent free from water.

In the second aspect of the invention the composition, the compounds of Formula (1) and salts thereof, the organic solvent in the mixture of water and organic solvent and the organic solvent free from water are as described and preferred in the first aspect of the invention.

It is especially preferred that the composition according to the second aspect of the invention is ink suitable for use in an ink-jet printer.

According to a third aspect of the invention there is provided a mono-azo compound of Formula (1) and salts thereof:

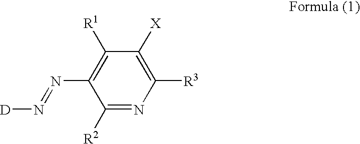

wherein:
D is an optionally substituted aryl group carrying at least one direct substituent selected from the group consisting of —SO₃H, —CO₂H, —PO₃H₂ or an optionally substituted nitrogen heteroaryl group carrying at least one direct substituent selected from the group consisting of —SO₃H, —CO₂H, —PO₃H₂;
R¹ is H: optionally substituted C₁₋₄alkyl: amino: —NR⁴R⁵; wherein R⁴ is H, optionally substituted alkyl, optionally substituted alkoxy, optionally substituted acyl, optionally substituted aryl, optionally substituted heterocyclyl; and R⁵ is optionally substituted alkyl, optionally substituted alkoxy, optionally substituted acyl, optionally substituted aryl, optionally substituted heterocyclyl: —OR⁶; wherein R⁶ is H or optionally substituted alkyl: or —SR⁷; wherein R⁷ is optionally substituted alkyl;
R² and R³ are each independently preferably amino: —NR⁴R⁵; wherein R⁴ is H, optionally substituted alkyl, optionally substituted alkoxy, optionally substituted acyl, optionally substituted aryl, optionally substituted heterocyclyl; and R⁵ is optionally substituted alkyl, optionally substituted alkoxy, optionally substituted acyl, optionally substituted aryl, optionally substituted heterocyclyl: hydroxy —OR⁶; wherein R⁶ is H or optionally substituted alkyl: or —SR⁷; wherein R⁷ is optionally substituted alkyl;
X is H; an electron withdrawing group selected from the group consisting of CN, CO₂H, CO₂R⁸, CON(R⁸)₂, COR⁸ and —SO₂N(R⁸)₂, wherein R⁸ is H, optionally substituted C₁₋₄alkyl, optionally substituted C₁₋₄alkoxy, optionally substituted C₁₋₄acyl, optionally substituted phenyl, optionally substituted heterocyclyl; or X together with R³ forms an optionally substituted five or six membered carbocycle:
   provided that the compound of Formula (1) is free from fibre reactive groups.

Preferences for R¹, R², R³, D and X in the compound of Formula (1) in the third aspect of the invention are as described in the first aspect of the invention.

A fourth aspect of the invention provides a composition comprising a mono-azo compound of Formula (1) and salts thereof, as defined in the third aspect of the invention, and water.

A fifth aspect of the present invention provides a material preferably paper, plastic, a textile, metal or glass, more preferably paper, an overhead projector slide or a textile material, especially paper more especially plain, coated or treated papers printed with a mom-azo compound of Formula (1) and salts thereof as described in the third aspect of the invention, a composition according to the second or fourth aspect of the invention or by means of a process according to the first aspect of the invention.

It is especially preferred that the printed material of the fifth aspect of the invention is a print on a photographic quality paper printed using a process according to the first aspect of the invention.

A sixth aspect of the present invention provides an ink-jet printer cartridge comprising a chamber and an ink suitable for use in an ink-jet printer wherein the ink is in the chamber and the ink is as defined in the second or fourth aspects of the present invention. The cartridge may contain a high concentration ink and a low concentration ink, as described in the second aspect of the invention, in different chambers.

The invention is further illustrated by the following Examples in which all parts and percentages are by weight unless specified otherwise.

### EXAMPLE 1

### Preparation of Dye 1

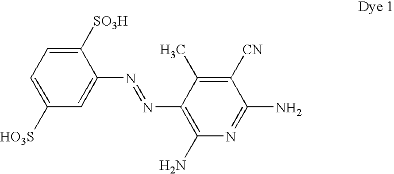

### Stage 1 (a): Preparation of the Intermediate (2,6-diamino-4-methylnicotinonitrile)

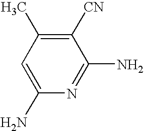

A solution of 2,6-dichloro-3-cyano-4-methylpyridine (0.35 mol, 65 g) in a mixture of 28% ammonium hydroxide solution (1000 ml) and 1-methylpyrrolidinone (100 ml) was stirred in an autoclave at 180-200° C. for 18 hours. The reaction mixture was allowed to cool, and the resultant solid was collected by filtration and dissolved in water (750 ml) and brought to pH 1 by the addition of concentrated hydrochloric acid. The solution was screened to remove a small amount of insoluble solid, the pH of the filtrate was adjusted to 8 with 48% sodium hydroxide solution and the precipitated solid was collected by filtration. The product was dried to give 30.5 g (59%) of a cream solid.

### Stage 1 (b): Preparation of the Title Compound

A solution of sodium nitrite (0.03 mol, 2.1 g) in water (25 ml) was added dropwise to a suspension of aniline-2,5-disulfonic acid (8.2 g, 0.025 mol) in a mixture of concentrated hydrochloric acid (20 ml) and water (150 ml) at 0-5° C. The solution was then stirred for 30 minutes to allow complete formation of the diazonium salt. After this time excess nitrous acid was quenched by addition of sulfamic acid. A solution of the product from Stage 1 (a) (0.025 mol, 3.7 g) in water (120 ml) was added to the above diazonium solution at 0-5° C., the pH of the reaction mixture was adjusted to 4-5 by the addition of sodium acetate and then allowed to warm to room temperature over 16 hours. The precipitated solid was collected by filtration and dissolved in water, dialysed to <100 μS and then dried at 60° C. to provide the pure product as a yellow solid (6.1 g, 59%).

### EXAMPLES 2 to 15

Dye examples 2 to 10 were prepared according to the above method for Example 1, wherein 2,6-diamino-4-methylnicotinonitrile was replaced with the compound shown in column 3 of Table 1. The final dye structure is shown in column 4 of Table 1.

Dye example 11 was prepared according to the above method for Example 1, wherein 2,5-disulfonaniline was replaced with the compound shown in column 2 of Table 1. The final dye structure is shown in column 4 of Table 1.

Dye examples 12 to 16 were prepared according to the above method for Example 1, wherein 2,5-disulfonaniline was replaced with the compound shown in column 2 of Table 1 and 2,6-diamino-4-methylnicotinonitrile was replaced with the compound shown in column 3 of Table 1. The final dye structure is shown in column 4 of Table 1.

TABLE 1

| Ex. | | |
|---|---|---|
| No. | A | B |
| 2 | 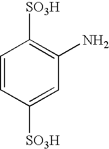 | 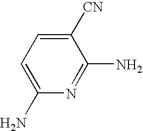 |
| 3 | 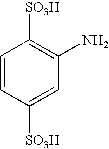 | 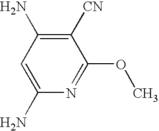 |
| 4 | 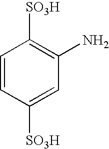 | 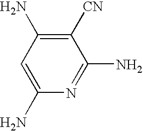 |
| 5 | 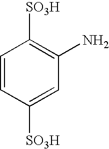 | 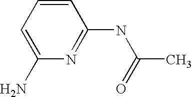 |
| 6 | 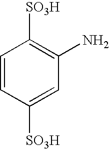 | 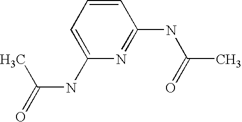 |
| 7 | 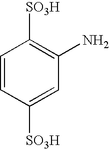 | 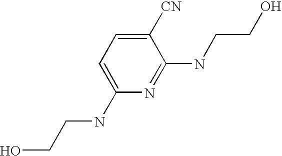 |
| 8 | 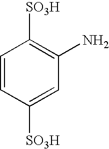 | 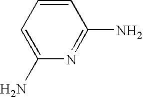 |
| 9 | 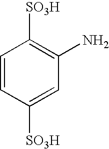 | 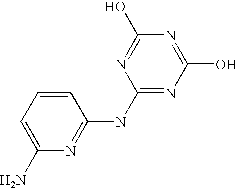 |
| 10 | 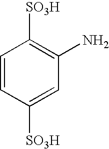 | 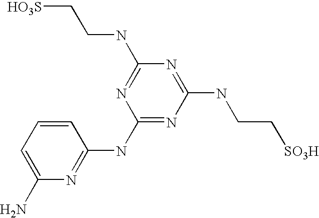 |
| 11 | 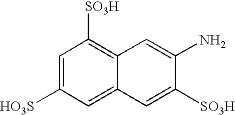 | 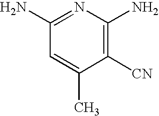 |
| 12 | 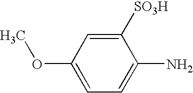 | 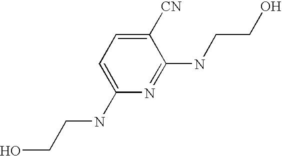 |
| 13 | 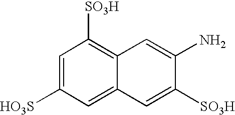 | 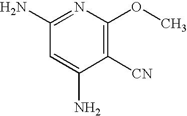 |
| 14 | 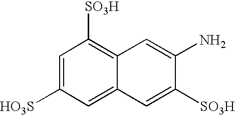 | 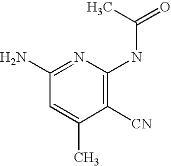 |
| 15 | 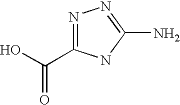 | 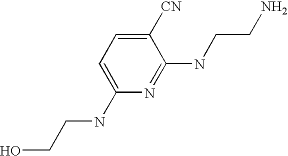 |
| 16 | 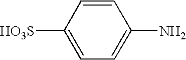 | 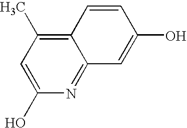 |

| Ex. | |
|---|---|
| No. | Dye |
| 2 | 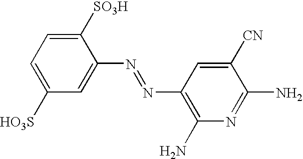 |
| 3 | 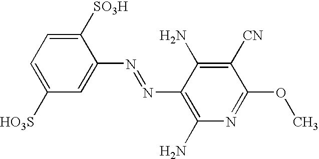 |
| 4 | 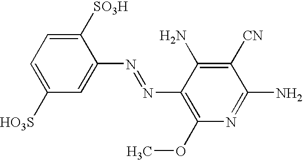 |
| 5 | 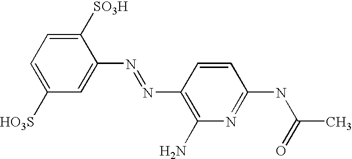 |
| 6 | 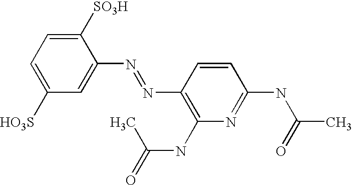 |
| 7 | 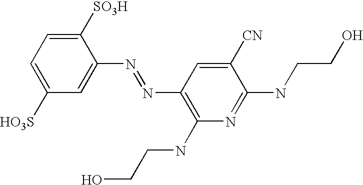 |
| 8 | 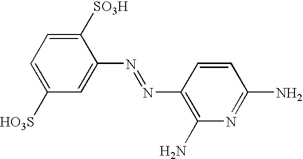 |
| 9 | 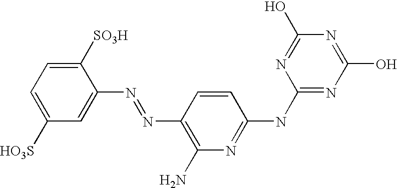 |
| 10 | 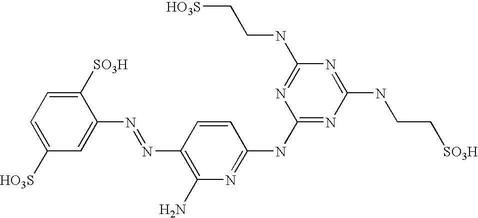 |
| 11 | 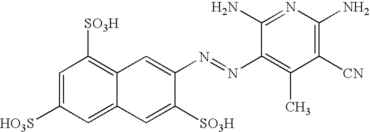 |
| 12 | 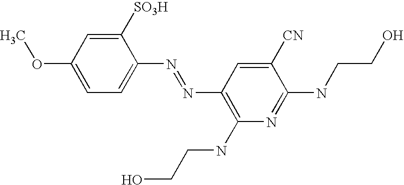 |
| 13 | 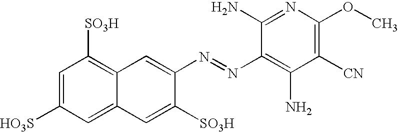 |
| 14 | 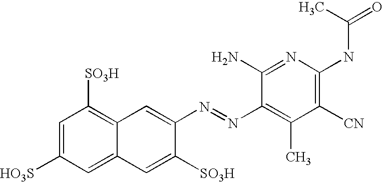 |
| 15 | 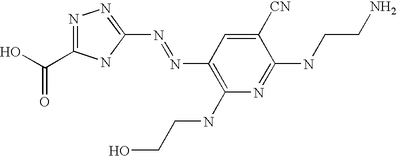 |
| 16 | 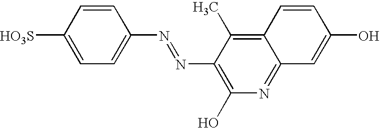 |

### COMPARATIVE EXAMPLE

### Preparation of

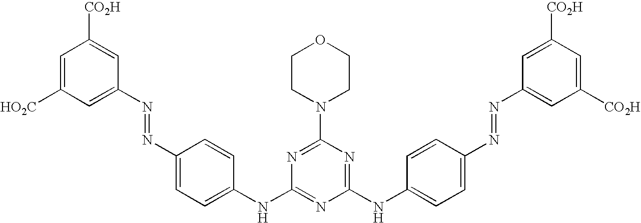

The dye of the Comparative Example was prepared as described in Example 10 of U.S. Pat. No. 5,347,301.

### EXAMPLE 17

### Preparation of the Example Inks and the Comparative Ink

Inks according to the invention and a Comparative Ink were prepared by dissolving 3 g of the dyes of Examples 2, 4, 7, 8 and 12 or the dye of the Comparative Example in 97 ml of a liquid medium consisting of 5 parts 2-pyrrolidone; 5 parts thiodiethylene glycol; 1 part Surfynol™ 465 and 89 parts water and adjusting the pH to between pH 8 to 9 with sodium hydroxide. Surfynol™ 465 is a surfactant from Air Products. The resultant inks are Example Inks 2, 4, 7, 8 and 12 and the Comparative Example Ink.

### EXAMPLE 18

### Ink-Jet Printing

The Example Inks and the Comparative Ink, prepared as described above, were then filtered through a 0.45 micron nylon filter and incorporated into empty print cartridges using a syringe.

These inks were printed (at 100%) using an inkjet printer onto Epson Premium Glossy Photo Paper (SEC PM).

### Print Evaluation

The chroma intensity of the resultant prints was measured using a Gretag spectrolino spectrophotometer set to the following parameters:

| | |
|---|---|
| Measuring Geometry | 0°/45° |
| Spectral Range | 380-730 nm |
| Spectral Interval | 10 nm |
| Illuminant | D65 |
| Observer | 2° (CIE 1931) |
| Density | Ansi A |
| External Filler | None. |

The chroma intensity of the prints obtained are shown below.

| Print of | Chroma Intensity at 100% print |
|---|---|
| Example Ink 2 | 102 |
| Example Ink 4 | 82 |
| Example Ink 7 | 114 |
| Example Ink 8 | 111 |
| Example Ink 12 | 115 |
| Comparative Example Ink | 76 |

### Further Inks

Further inks may be prepared according to Tables A and B wherein the dye described in the first column is the dye made in the above example of the same number. Numbers quoted in the second column onwards refer to the number of parts of the relevant ingredient and all parts are by weight. The inks may be applied to paper by thermal or piezo ink-jet printing.

The following abbreviations are used in Tables A and B:

PG=propylene glycol

DEG=diethylene glycol

NMP=N-methylpyrrolidone

DMK=dimethylketone

IPA=isopropanol

MeOH=methanol

2P=2-pyrrolidone

MIBK=methylisobutyl ketone

P12=propane-1,2-diol

BDL=butane-2,3-diol

CET=cetyl ammonium bromide

PHO=Na₂HPO₄ and

TBT=tertiary butanol

TDG=thiodiglycol

TABLE A

| | Dye | | | | | | | Na | | | | |
|---|---|---|---|---|---|---|---|---|---|---|---|---|
| Example | Content | Water | PG | DEG | NMP | DMK | NaOH | Stearate | IPA | MEOH | 2P | MIBK |
| 1 | 2.0 | 80 | 5 | | 6 | 4 | | | | | 5 | |
| 2 | 3.0 | 90 | | 5 | 5 | | 0.2 | | | | | |
| 3 | 10.0 | 85 | 3 | | 3 | 3 | | | | 5 | 1 | |
| 4 | 2.1 | 91 | | 8 | | | | | | | | 1 |
| 5 | 3.1 | 86 | 5 | | | | | 0.2 | 4 | | | 5 |
| 6 | 1.1 | 81 | | | 9 | | 0.5 | 0.5 | | | 9 | |
| 7 | 2.5 | 60 | 4 | 15 | 3 | 3 | | | 6 | 10 | 5 | 4 |
| 8 | 5 | 65 | | 20 | | | | | 10 | | | |
| 9 | 2.4 | 75 | 5 | 4 | | 5 | | | | 6 | | 5 |
| 10 | 4.1 | 80 | 3 | 5 | 2 | 10 | | 0.3 | | | | |
| 11 | 3.2 | 65 | | 5 | 4 | 6 | | | 5 | 4 | 6 | 5 |
| 12 | 5.1 | 96 | | | | | | | | 4 | | |
| 13 | 10.8 | 90 | 5 | | | | | | 5 | | | |
| 14 | 10.0 | 80 | 2 | 6 | 2 | 5 | | | 1 | | 4 | |
| 15 | 1.8 | 80 | | 5 | | | | | | | 15 | |
| 16 | 2.6 | 84 | | | 11 | | | | | | 5 | |
| 1 | 3.3 | 80 | 2 | | | 10 | | | | 2 | | 6 |
| 1 | 12.0 | 90 | | | | 7 | 0.3 | | 3 | | | |
| 1 | 5.4 | 69 | 2 | 20 | 2 | 1 | | | | | 3 | 3 |
| 1 | 6.0 | 91 | | | 4 | | | | | | 5 | |

TABLE B

| | Dye | | | | | | | | | | | |
|---|---|---|---|---|---|---|---|---|---|---|---|---|
| Example | Content | Water | PG | DEG | NMP | CET | TBT | TDG | BDL | PHO | 2P | PI2 |
| 1 | 3.0 | 80 | 15 | | | 0.2 | | | | | 5 | |
| 2 | 9.0 | 90 | | 5 | | | | | | 1.2 | | 5 |
| 3 | 1.5 | 85 | 5 | 5 | | 0.15 | 5.0 | 0.2 | | | | |
| 4 | 2.5 | 90 | | 6 | 4 | | | | | 0.12 | | |
| 5 | 3.1 | 82 | 4 | 8 | | 0.3 | | | | | | 6 |
| 6 | 0.9 | 85 | | 10 | | | | | 5 | 0.2 | | |
| 7 | 8.0 | 90 | | 5 | 5 | | | 0.3 | | | | |
| 8 | 4.0 | 70 | | 10 | 4 | | | | 1 | | 4 | 11 |
| 9 | 2.2 | 75 | 4 | 10 | 3 | | | | 2 | | 6 | |
| 10 | 10.0 | 91 | | | 6 | | | | | | 3 | |
| 11 | 9.0 | 76 | | 9 | 7 | | 3.0 | | | 0.95 | 5 | |
| 12 | 5.0 | 78 | 5 | 11 | | | | | | | 6 | |
| 13 | 5.4 | 86 | | | 7 | | | | | | 7 | |
| 14 | 2.1 | 70 | 5 | 5 | 5 | 0.1 | 0.2 | 0.1 | 5 | 0.1 | 5 | |
| 15 | 2.0 | 90 | | 10 | | | | | | | | |
| 16 | 2 | 88 | | | | | | 10 | | | | |
| 1 | 5 | 78 | | | 5 | | | 12 | | | 5 | |
| 1 | 8 | 70 | 2 | | 8 | | | 15 | | | 5 | |
| 1 | 10 | 80 | | | | | | 8 | | | 12 | |
| 1 | 10 | 80 | | 10 | | | | | | | | |

1. A process for printing an image on a substrate by means of an ink-jet printer which comprises applying thereto a composition comprising a liquid medium and a mono-azo compound of Formula (1) and salts thereof: 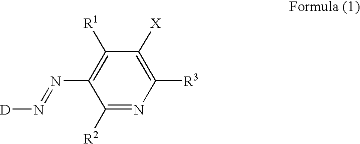
wherein:
D is an optionally substituted aryl group carrying at least one direct substituent selected from the group consisting of —SO₃H, —CO₂H, —PO₃H₂ or an optionally substituted nitrogen heteroaryl group carrying at least one direct substituent selected from the group consisting of —SO₃H, —CO₂H, —PO₃H₂;
R¹ is H: optionally substituted alkyl: amino: —NR⁴R⁵; wherein R⁴ is H, optionally substituted alkyl, optionally substituted alkoxy, optionally substituted acyl, optionally substituted aryl, optionally substituted heterocyclyl; and R⁵ is optionally substituted alkyl, optionally substituted alkoxy, optionally substituted acyl, optionally substituted aryl, optionally substituted heterocyclyl:
—OR⁶; wherein R⁶ is H or optionally substituted alkyl: or —SR⁷; wherein R⁷ is optionally substituted alkyl;
R² and R³ are each independently preferably amino: —NR⁴R⁵; wherein R⁴ is H, optionally substituted alkyl, optionally substituted alkoxy, optionally substituted acyl, optionally substituted aryl, optionally substituted heterocyclyl; and R⁵ is optionally substituted alkyl, optionally substituted alkoxy, optionally substituted acyl, optionally substituted aryl, optionally substituted heterocyclyl: hydroxy —OR⁶; wherein R⁶ is H or optionally substituted alkyl- or —SR⁷; wherein R⁷ is optionally substituted alkyl;
X is H, or an electron withdrawing group, or X together with R³ forms an optionally substituted five or six membered carbocycle.

2. A process according to claim 1 wherein D is optionally substituted phenyl or optionally substituted napthyl carrying at least one direct substituent selected from the group consisting of —SO₃H, —CO₂H, —PO₃H₂.

3. A process according to claim 1 or wherein D is optionally substituted phenyl or optionally substituted napthyl carrying 1 to 3-SO₃H substituents.

4. A process according to claim 1 wherein D is a 1,3,4-triazole carrying at least one direct substituent selected from the group consisting of —SO₃H, —CO₂H, —PO₃H₂.

5. A process according to claim 1 wherein R¹ is H, amino, NHR⁵ wherein R⁵ is optionally substituted C₁₋₄alkyl or optionally substituted C₁₋₄acyl.

6. A process according to claim 1 wherein R² and R³ are each independently: amino; NHR⁵ wherein R⁵ is optionally substituted C₁₋₄alkyl or optionally substituted C₁₋₄acyl; —OR⁶; wherein R⁶ is H or optionally substituted C₁₋₄alkyl.

7. A process according to claim 1 wherein X is selected from the group consisting of CN, CO₂H, CO₂R⁸, CON(R⁸)₂, COR⁸ and —SO₂N(R⁸)₂ wherein R⁸ is independently H, optionally substituted C₁₋₄alkyl, optionally substituted C₁₋₄alkoxy, optionally substituted C₁₋₄acyl, optionally substituted phenyl, optionally substituted heterocyclyl.

8. A process according to claim 1 wherein X together with R³ forms an optionally substituted six membered carbocycle.

9. A process according to claim 1 wherein the compounds of Formula (1) and salts thereof are free from fibre reactive groups.

10. A process according to claim 1 wherein the liquid medium comprises a mixture of water and organic solvent or organic solvent free from water.

11. (canceled)

12. A composition comprising a compound of Formula (1) and salts thereof, as defined in claim 1 and a liquid medium that comprises a mixture of water and organic solvent or organic solvent free from water.

13. A composition according to claim 12 which is ink suitable for use in an inkjet printer.

14. A mono-azo compound of Formula (1) and salts thereof: 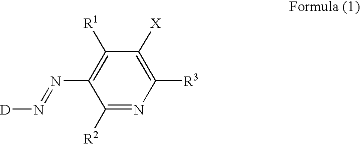
wherein:
D is an optionally substituted aryl group carrying at least one direct substituent selected from the group consisting of —SO₃H, —CO₂H, —PO₃H₂ or an optionally substituted nitrogen heteroaryl group carrying at least one direct substituent selected from the group consisting of —SO₃H, —CO₂H, —PO₃H₂;
R¹ is H: optionally substituted C₁₋₄alkyl: amino: —NR⁴R⁵; wherein R⁴ is H, optionally substituted alkyl, optionally substituted alkoxy, optionally substituted acyl, optionally substituted aryl, optionally substituted heterocyclyl; and R⁵ is optionally substituted alkyl, optionally substituted alkoxy, optionally substituted acyl, optionally substituted aryl, optionally substituted heterocyclyl: —OR⁶; wherein R⁶ is H or optionally substituted alkyl: or —SR⁷; wherein R⁷ is optionally substituted alkyl;
R² and R³ are each independently preferably amino: —NR⁴R⁵; wherein R⁴ is H, optionally substituted alkyl, optionally substituted alkoxy, optionally substituted acyl, optionally substituted aryl, optionally substituted heterocyclyl; and R⁵ is optionally substituted alkyl, optionally substituted alkoxy, optionally substituted acyl, optionally substituted aryl, optionally substituted heterocyclyl: hydroxy —OR⁶; wherein R⁶ is H or optionally substituted alkyl: or —SR⁷; wherein R⁷ is optionally substituted alkyl;
X is H; an electron withdrawing group selected from the group consisting of CN, CO₂H, CO₂R⁸, CON(R⁸)₂, COR⁸ and —SO₂N(R⁸)₂, wherein R⁸ is H, optionally substituted C₁₋₄alkyl, optionally substituted C₁₋₄alkoxy, optionally substituted C₁₋₄acyl, optionally substituted phenyl, optionally substituted heterocyclyl; or X together with R³ forms an optionally substituted five or six membered carbocycle:
provided that the compound of Formula (1) is free from fibre reactive groups.

15. A mono-azo compound and salts thereof according to claim 14 wherein D is optionally substituted phenyl or optionally substituted napthyl carrying at least one direct substituent selected from the group consisting of —SO₃H, —CO₂H, —PO₃H₂.

16. A mono-azo compound and salts thereof according to claim 14 wherein D is optionally substituted phenyl or optionally substituted napthyl carrying 1 to 3-SO₃H substituents.

17. A mono-azo compound and salts thereof according to claim 14 wherein R¹ is H, amino, NHR⁵ wherein R⁵ is optionally substituted C₁₋₄alkyl or optionally substituted C₁₋₄acyl.

18. A composition comprising a mono-azo compound of Formula (1) and salts thereof, as defined in claim 14, and water.

19. A material printed with a mono-azo compound of Formula (1) and salts thereof as described in claim 14.

20. A material according to claim 19 which is a print on a photographic quality paper.

21. An ink-jet printer cartridge comprising a chamber and an ink suitable for use in an ink-jet printer wherein the ink is in the chamber and the ink is as defined in claim 13.
